(19) United States
(12) Patent Application Publication
(10) Pub. No.: US 2008/0187906 A1
(43) Pub. Date: Aug. 7, 2008

(54) Method And Kit For Assessing The Risk Of Cardiovascular Pathologies With Atheromatous Etiology

(75) Inventors: Francesco Cipollone, Montesilvano (IT); Andrea Mezzetti, Francavilla (IT); Stefano Martinotti, Roma (IT)

Correspondence Address: KNOBBE MARTENS OLSON & BEAR LLP, 2040 MAIN STREET, FOURTEENTH FLOOR, IRVINE, CA, 92614, US

(73) Assignee: Gene Design & Development S.R.I., Pescara (IT)

(21) Appl. No.: 10/567,486

(22) PCT Filed: Aug. 4, 2004
(86) PCT No.: PCT/EP04/51711

§ 371 Date: Feb. 6, 2006

(30) Foreign Application Priority Data
Aug. 5, 2003 (IT) MI2003A001607

(51) Int. Cl. C12Q 1/68

(52) U.S. Cl. 435/6

(57) ABSTRACT

The invention relates to a molecular method for in vitro assessment of the predisposition of a subject to develop cardiovascular pathologies, associated with rupture of an atherosclerotic plaque. The method is characterized by the identification of a polymorphism in the COX-2 gene promotor, using genomic DNA sample(s) of the subject or a group of subjects. Moreover the invention relates to a kit for detection of such polymorphism.

### FIELD OF THE INVENTION

The field of invention pertains to molecular methods for diagnosis and prognosis of cardiovascular diseases.

### STATE OF THE ART

Myocardial infarction and stroke are caused mainly by the rupture of unstable atherosclerotic plaques formed in coronary and carotid arteries.

They represent the main cause of death and disability in western countries. It is estimated that, only in United States, approximately 62 million persons are affected by cardiovascular pathologies, and, in the year 2000, approximately one million deaths (39% of the total) have been attributed to these pathologies. There are reasons to consider that a similar incidence holds for our country. Epidemiological and randomized studies have provided solid evidences that infarction and stroke can be prevented by treating classical risk factors, such as diabetes, hypertension, smoking habit, hypercholesterolemy, obesity. Nevertheless, controlling these factors has reduced the incidence of infarction or stroke but did not abolish the risk for these diseases. This suggests the existence of an as yet unclear hereditary component in infarction and stroke.

Therefore the need for predictive tests of the genetic risk to develop such diseases is highly felt in the field.

Recently, Papafili and collaborators reported the identification of two variants of the human cyclooxygenase-2 (COX-2) gene promotor: a G→C mutation in position −765 and a C+G mutation in position −490 (Papafili et al., Arterioscler Thromb Vasc Biol. 2002; 22:1631-1636). The presence of the former variant (cytosine or C in position −765) has been associated with reduced expression of the COX-2 gene and with lower serum levels of C-reactive protein (CRP) as compared to the respective levels detected in subjects undergone coronary by-pass and carrying a G nucleotide at the same position. Therefore, Papafili hypothesizes that such polymorphism may represent a diagnostic index of special relevance in the acute inflammatory response.

### SUMMARY OF THE INVENTION

The invention relates to a method for in vitro assessment of the predisposition of a subject to develop cardiovascular pathologies, associated with rupture of an atherosclerotic plaque. The method is characterized by the identification, in a genomic DNA sample of such subject, of the nucleotide at position 436 of seq IDN1.

The method is performed preferably by means of sequencing, endonuclease digestion with restriction enzymes, selective hybridization with oligonucleotides specific for said polymorphism in the human COX-2 gene promotor, or by Real Time PCR. In case the method is based on endonuclease digestion, this is preceded by PCR amplification with oligonucleotides comprising seq ID NO 3 and 4. Moreover the invention comprises a kit to carry out the detection method.

### DESCRIPTION OF THE FIGURES

FIG. 1. Visualization, by means of electrophoresis, of the restriction profile after enzymatic digestion with the Fau I restriction enzyme.

The fragment amplified from the COX-2 gene promotor region, using the oligonucleotides seq ID NO 3 and ID NO 4 (Cox-F and Cox-R), consists of 231 bp. It contains the consensus sequence (CCCGCC) recognized by the enzyme Fau I. This enzyme cuts only the wild type sequence carrying a G nucleotide at position −765 of the promotor. After enzymatic digestion, the DNA solution is subjected to electrophoretic run in polyacrylamide gel. In the case of wild type sequence (CCCGCC), two fragments, respectively of 124 and 107 bp, are produced by endonucleolytic cut. Only the intact 231 bp fragment is obtained in the case of the mutated sequence (CCCCCC), carrying a C rather than a G nucleotide. For visualization of bands, the gel is stained with an ethidium bromide solution and then irradiated with a UV ray source. Samples A, B and D are homozygous for the COX-2 G/G −765 polymorphism; sample C is C/C homozygous; samples E,F are G/C heterozygous.

FIG. 2: COX-2 expression in atherosclerotic plaques

The picture shows the expression of the COX-2 gene in sections of atherosclerotic plaques, detected by means of an immunohistochemical method based on specific antibodies for the COX-2 enzyme. Carriers of −765 polymorphism in homozygous configuration (G/G) show a more intense staining compared to the other subjects, either heterozygous (G/C) or homozygous (C/C).

FIG. 3: Expression of metalloproteinases in atherosclerotic plaques.

The picture shows the expression of metalloproteinases 2 (MMP2) and 9 (MMP9) in sections of atherosclerotic plaques, detected by means of an immunohistochemical method based on anti-metalloproteinase antibodies. The expression turns out to be higher in individuals homozygous (G/G) for the −765 polymorphism compared to heterozygous (G/C) and homozygous (C/C).

### DETAILED DESCRIPTION OF THE INVENTION

### Definitions

For the purpose of the present invention, the terms listed below have the following meaning:

Allele: alternative form of a gene. The alleles of a given gene reside in the same position on homologous chromosomes.

### BMI: Body Mass Index.

COX-2 (cyclooxygenase 2): product of the gene partially corresponding to the gene region whose sequence is reported in the GenBank with accession n^{o} AF276953, (Homo Sapiens PTGS2 gene) and corresponding to SEQ ID NO 1.
Controls: subjects with high cardiovascular risk but without previous myocardial infarction or ictus cerebr.
Heterozygous: subject carrying different alleles in the same locus of the two homologous chromosomes.
Genotyping: determination of the alleles of a chromosome in a given subject.
Homozygous: subject carrying identical alleles in the same locus of the two homologous chromosomes.
Patients: subjects with high cardiovascular risk and prior myocardial infarction or ictus cerebri (patients and controls of this study have undersigned an informed consent before each examination).
Vulnerable atherosclerotic plaques: plaques that are considered unstable due to the presence of an intense inflammatory infiltrate capable of provoking rupture with time.
Polymorphism: situation in which, in a population of subjects, two or more alleles of a locus show differences in at least one nucleotide.
Polymorphism at position 436 of seq IDN1 (COX-2 gene): referred to the polymorphism in the COX-2 gene promotor at position −765 from the transcription start site, described by Papafili et al., Arterioscier Thromb Vasc Biol. 2002; 22:1631-1636. In the present invention, it is defined as wild type sequence the one presenting a G in position n^{o} 436 of seq IDN1 and comprised within the sequence (CCCGCC).

### Description

The present invention is based on the clinical confirmation of the existence of a relationship between the C/G polymorphism in the COX-2 promotor in position corresponding to nucleotide 436 in SEQ. ID NO 1 and the instability of the atherosclerotic plaque. The direct consequence of such instability is a higher risk of cardiovascular pathologies associated with rupture and erosion of vulnerable atherosclerotic plaques, in particular of myocardial infarction and stroke.

Therefore, according to a first aspect, the invention relates to a method for in vitro diagnosis of the predisposition of a subject to develop cardiovascular pathologies associated with rupture or erosion of vulnerable atherosclerotic plaques, in particular myocardial infarction and ictus. The method essentially comprises the identification of the nucleotide at position −765 (corresponding to nucleotide 436 of SEQ. ID NO 1) from the transcription start site of the cyclooxygenase-2 (COX-2) gene on a sample of genomic DNA from a biological sample of such subject. The biological sample is preferably blood, saliva, biopsies, urine. Even more preferably the sample is blood.

The diagnostic and prognostic value of the invention is supported by a study on a sample of 1441 subjects recruited in Italian centers, indicating that the group of patients that did not suffer myocardial infarction or stroke carries, on at least one allele, the polymorphic variant corresponding to the C nucleotide in position 436 of seq IDN1 with a frequency significantly higher than in the group of patients with previous myocardial infarction or stroke. Samples were balanced with respect to age, BMI, or conventional risk factors for cardiovascular pathologies such as: cigarette smoking, obesity, hypertension, hypercholesterolemy and diabetes.

The study data are presented in table 2: in particular, it is evidentiated that the frequency of heterozygous subjects for the polymorphism in position −765 is at least 2 fold higher in the controls than in patients (43.1% vs 17.9%) and that the frequency of homozygous subjects for C/C polymorphism in position −765 (that is presence of C on both alleles) is at least five times higher in controls than in patients (6.2% vs 1.1%).

Moreover the clinical study allowed to verify in situ the condition of atherosclerotic plaques, confirming that COX-2 enzyme expression is significantly higher in carriers of the G/G −765 polymorphism (hence in homozygous configuration) and decreases in heterozygous and in homozygous for C/C polymorphism (8576±176, 5132±142, 3059±117, respectively). This situation correlates with the expression profile of the COX-2 enzyme in macrophages isolated from atherosclerotic plaques, as well as in monocytes isolated from peripheral blood.

Furthermore the expression of matrix metalloproteinases (MMP) 2 and 9, as assessed in situ by immunohistochemistry, western-blot and zymography on atherosclerotic plaques, is elevated in carriers of G/G polymorphism in homozygous configuration, compared to G/C heterozygous and to C/C homozygous. In particular, Western blot values obtained for MMP-2 turn out to be: G/G homozygous 7165±134 vs G/C 5121±78 and C/C 3978±67 arbitrary densitometric units (DU); for MMP-9, the values were 8090±187 vs 6012±102 and 4765±95 DU; mean ±SD; P<0.0001). By zymography, the amount of active enzyme is for MMP-2: G/G homozygous 3012±134, G/C heterozygous 1898±104 and C/C homozygous 1342±54 DU; for MMP-9: 3867±165 vs 2011±89 and 1223±65 DU; mean ±SD; P<0.0001). These data appear to correlate well with the hypothesis that the instability of atherosclerotic plaques is due directly to a proteolytic action exerted by enzymes such as matrix metalloproteinases activated by expression of the COX-2 gene, which are expressed at a higher level, like the COX-2 enzyme, in carriers of the G −765 polymorphism compared to carriers of C −765 polymorphism.

Others have already emphasized that the −765 G/C polymorphism is positioned in a region that is very close to a consensus sequence for the transcription factor SP1 (GGGAGG and the variants GGGCCC and CCCGCC, as described in Xu Q et al., J Biol Chem 2000; 275:24583-24589), therefore may have a direct effect on the expression levels of such gene.

Moreover, in the present study it has been confirmed that also the levels of hsCRP (high sensitivity C-Reactive Protein, described in Ridker et al., Circulation, 1998, 97:425-428) are significantly lower in carriers of C polymorphism compared to subjects not carrying the polymorphism (0.78±0.1 vs 2.56±0.4), in a subgroup of approximately 200 subjects.

Verification of identity of the nucleotide at position −765 for prognostic purpose, to assess the risk or predisposition to develop pathologies such as myocardial infarction or stroke, due to rupture of atherosclerotic plaques, is carried out by one of the following techniques: direct sequencing of the region comprising such polymorphism, endonucleolytic digestion with restriction enzymes, selective hybridization with oligonucleotides specific for polymorphism in position −765 of the human COX-2 gene promotor. Several techniques, well known in the art, can be used to identify in genomic DNA the presence of the mutation according to the method of the invention. Suitable techniques are, for example, the techniques based on acquisition or loss of restriction enzyme recognition sites (Kan et al., Lancet: 910-912, 1978), hybridization techniques with allele-specific oligonucleotide probes (ASO) (Wallace et al., Nucl Acids Res 6: 3543-3557, 1978) among which, for example, hybridization with oligonucleotides immobilized on filters (Saiki et al., PNAS USA 86: 6230-6234, 1989) or micro-chips (Chee et al., Science 274:610-614, 1996) and oligonucleotide arrays (Maskos et al., Nucl Acids Res 21: 2269-2270, 1993), allele-specific PCR (Newton et al., Nucl Acid Res 17:2503-2516, 1989), Mismatch Repair Detection (MRD) (Faham and Cox, Genome Res: 474-482, 1995), Single-Strand Conformational Polymorphism Analysis (Ravnik-Glavac et al., Hum. Mol. Gen. 3: 801, 1994), gel electrophoresis in denaturing gradient (Guldberg et al., Nucl. Acids Res. 22: 880, 1994), Hot Cleavage (Cotton et al., Proc. Natl. Acad Sci USA 85: 4397, 1988), Genetic Bit Analysis (GBA) (Nikiforov et al., Nucl Acid Res 22:4167-4175, 1994), primer-ligation assay (OLA) (Landergen et al., Science 241: 1077, 1988), allele specific ligation chain reaction (LCR) (Barrany PNAS USA 88:189-193, 1991), gap-LCR (Abravaya et al., Nucl Acids Res 23: 675-682, 1995), Real-Time PCR techniques (Chen Y Y, Int J Oncol. 2003 September; 23(3):737-44) and sequencing techniques. Particularly preferred techniques, for identification of −765 polymorphism according to the method of invention, are chosen among those based on acquisition or loss of restriction enzyme recognition sites, allele specific PCR, hybridization techniques, Real-Time PCR or direct sequencing techniques after amplification of DNA fragments corresponding to the promotor region of the COX-2 gene and comprising position −765.

Method and kit of the invention make possible to evaluate, in an apparently healthy subject or group of subjects, the risk to develop cardiovascular pathologies due to the rupture of atherosclerotic plaques. According to a preferred embodiment, the method comprises the following steps:
a) genomic DNA extraction from a biological sample of such subject/subjects,
b) amplification by Polymerase Chain Reaction with oligonucleotides or primers suitable for amplification of a DNA fragment comprising position −765,
c) enzymatic digestion of such amplified fragment with restriction enzymes capable of discriminating between sequences carrying the C −765 polymorphism, comprised in the hexanucleotide sequence CCCCCC, and the G −765 polymorphism, comprised in the hexanucleotide sequence CCCGCC,
d) electrophoretic separation of the restriction mixture comprising the restriction fragments produced or of the undigested amplified fragment, or of both, and analysis of the length of such fragments, where in particular the acquisition of Aci I or Fau I restriction enzyme sites (CCGC//GGCG or CCCGC(N)4//GGGCG(N)6, respectively) indicates the presence at position −765 of a guanine (G) that correlates with a higher risk factor to develop cardiovascular diseases, in particular myocardial infarction and stroke, whereas the loss of such restriction site correlates with a lower risk index.

In the endonucleolytic digestion with restriction enzymes specific for the 4-12 base sequence comprising the polymorphic nucleotide, both the enzyme Fau and the enzyme Aci are used. However, the former is preferred because more stable and specific.

According to a preferred embodiment, the amplification in b) is carried out with oligonucleotide primers that amplify a genomic DNA fragment, directly or after extraction from the biological sample, the length of which is preferably shorter than 1000 nucleotides, preferably less than 500 nucleotides, even more preferably less than 300 nucleotides, and that comprises the polymorphism at position −765. Suitable oligonucleotides for amplification of a fragment of appropriate dimension can be designed according to methods known in the art, given that the sequence of the COX-2 gene promotor region is known (GenBank, accession number: AF276953), but must be opportunely validated for amplification specificity in various biological matrices. Such primers are preferably oligonucleotides at least partially identical, i.e. comprising a sequence of at least 10 consecutive nucleotides, to the oligonucleotides with sequence ID NO 3 and ID NO 4. Even more preferably, the oligonucleotides for PCR (Polymerase Chain Reaction) amplification of the fragment comprising the −765 polymorphism are the oligonucleotides SEQ. ID NO 3 and SEQ. ID NO 4. The fragment generated by amplification with these oligos, 231 nucleotides in length, is preferably digested with the restriction enzyme Fau I and, subsequently, is preferably analyzed by agarose or polyacrylamide gel electrophoresis.

According to the method and to the prognostic and/or diagnostic kits of the invention, the presence of a cytosine at position −765 of the COX-2 gene promotor, hence the absence of the recognition sequence for the above specific restriction enzymes on at least one allele of the DNA of a subject or of a group of subjects, correlates with a risk index for predisposition to cardiovascular diseases that is lower than the index associated to the presence of a guanosina (G) at position −765 on both alleles, hence in homozygous configuration. The presence of guanosine (G) at position −765, and therefore the presence of the recognition sequence for the specific restriction enzymes indicated above, correlates with a condition of higher risk to develop cardiovascular pathologies, in particular coronaropathies and pathologies of the carotid arteries, myocardial infarction, angina pectoris, acute coronary syndromes, stroke, transient ischemic attack (TIA) and peripheral arteriopathy, and all trombophylic syndromes in general. Therefore, the identification of such nucleotide at position −765, determined from endonucleolytic fragments according to the method of the invention, together with physician's assessment of other medical parameters, provides an intermediate indication about the suitability of an adequate preventive therapy for such diseases.

Moreover the genotyping data, i.e. the characterization of the nucleotide at position −765 of the COX-2 gene promotor, is associated with lower expression of the enzyme cyclooxygenase-2 under both basal conditions and after stimulation, thus allowing the assessment of sensitivity to non steroidal anti-inflammatory drugs (FANS), and in particular with specific cyclooxygenase-2 inhibitors, which also concur to reduce the levels of cyclooxygenase activity particularly in lymphocytes and macrophages.

According to a further aspect, the invention relates to a kit for genotyping position −765 in the cyclooxygenase-2 (COX-2) promotor according to the preferred embodiment of the method (acquisition or loss of a restriction site), in order to assess the sensitivity to treatment with non steroidal anti-inflammatory drugs (FANS), in particular specific cyclooxygenase-2 inhibitors.

Moreover, in a preferred aspect, the invention relates to a prognostic kit in order to evaluate, even in apparently healthy subjects, the risk factor for cardiovascular diseases caused by rupture of unstable atherosclerotic plaques, such as coronaropathies that cause myocardial infarction, angina pectoris, acute coronary syndromes, stroke, transient ischemic attack (TIA) and peripheral arteriopathy, based on the method and according to its preferred embodiment (acquisition or loss of a restriction site).

In any case, when the method or kit to determine the risk of cardiovascular pathologies by genotypization of nucleotide 436 of the COX-2 promotor is based on PCR amplification with suitable oligonucleotides, preferably with seq IDN3 and 4 according to the invention and on the subsequent digestion of the PCR product with restriction endonuclease, the cleavage of wild type sequence (CCCGCC, where G is at position −765) with Fau I or Aci I produces two fragments of approximately 120 and 110 bp. The presence of at least one allelic copy of these two fragments correlates with a higher risk index for developing cardiovascular pathologies or with a higher sensitivity to the therapy with FANS, in particular those of the Coxib (COX-2 inhibitors) class. In the case of the mutated sequence (CCCCCC), carrying the nucleotide C instead of G, where the digestion does not occur and the 231 bp fragment remains intact, the presence of at least one allelic copy of such fragment correlates with a lower risk index for developing cardiovascular pathologies or with lower sensitivity to the therapy with FANS, particularly those of the Coxib (COX-2 inhibiting) class.

Preferably this kit is in the form of a box or container comprising: a tube containing an oligonucleotide whose sequence is at least partially identical to SEQ ID NO 3, i.e. at least 10 consecutive nucleotides identical to oligonucleotides with sequence ID NO 3 and ID NO 4, or even more preferably the oligonucleotide with seq ID NO 3; a tube containing an oligonucleotide whose sequence is at least partially identical to SEQ ID NO 4, i.e. at least 10 consecutive nucleotides identical to oligonucleotides with sequence SEQ ID N. 4, or even more preferably is the oligonucleotide with seq ID NO 4; optionally, a restriction enzyme that can discriminate the C −765 polymorphism in the hexanucleotide sequence CCCCCC from the polymorphism in the hexanucleotide sequence CCCGCC. Such enzyme is preferably chosen among: the enzyme Fau I and Aci I, even though the enzyme Fau I is preferred because it is more specific and stable, in that it recognizes, for endonucleolytic cleavage, the nucleotide sequence CCCGC(N)4↓//GGGCG(N)6↓ (Fau I) rather than the teatranucleotide sequence C↓CGC//GGC↓G (Aci I). Optionally, the kit includes a DNA molecular weight standard and/or a tube containing a suitable concentration of magnesium ions in buffered solution and/or deoxynucleotides triphosphate.

A further aspect of the invention relates to the use of genotyping of the nucleotide at position −765 of the COX-2 gene promotor for preparation of prognostic tests, essentially based on the described method, for cardiovascular pathologies selected among: coronaropathies, pathologies of carotid arteries, myocardial infarction, angina pectoris, acute coronary syndromes, myocardial revascularization by means of coronary by-pass or angioplasty, stroke, transient ischemic attack (TIA), peripheral arteriopathy, trombophylic syndromes.

Moreover a further aspect consists in the use of genotyping the nucleotide in position −765 of the COX-2 gene promotor for preparation of diagnostic tests of the sensitivity to therapy with not steroidal anti-inflammatory drugs (FANS), essentially based on the described method.

### EXPERIMENTAL PART

### Example 1

### DNA Extraction from Peripheral Blood for Genotyping

Approximately 5 ml of peripheral blood were withdrawn from the study sample subjects and collected in hemochromocytometric test-tubes containing EDTA/citrate and lacking heparin solution. Part of the content, approximately 0.5 ml, was then transferred to “DNase free” test-tubes and mixed with one volume of lysis solution (lysis solution according to the extraction protocol of the Diatech Company, Jesi-Ancona, Italy); it was then subjected to shaking for few seconds by means of a vortex and is centrifuged at ambient temperature. The supernatant was then discarded and the pellet was resuspended in 1 ml of lysis solution in order to separate, by further centrifugation, the DNA pellet from the products of hemolysis that were present in solution.

The pellet purified as above was then redissolved in buffer containing Proteinase K and a low concentration (0.1%) of sodium lauryl sulfate (SLS) in order to solubilize further the DNA and to remove proteinaceous and lipid contaminants. Following further purification, the DNA pellet was dissolved in approximately 50 l of water buffered with 10 mM Tris pH 7.6 and 0.1 mM EDTA.

A PCR reaction was prepared according to the standard protocol supplied by the manufacturer. The protocol outline was as follows:

100 ng DNA template (genomic DNA or cDNA sample)
100 μM final dNTP
10 pmoles of COX-F and COX-R (corresponding to seq ID NO 3 and 4)
1× Taq buffer (Perkin-Elmer Taq buffer, composition without Mg)
1.5 mM Mg⁺⁺ (final concentration)
2.5 Units of Taq polymerase

| N^{o} cycles | Temperature | Time |
|---|---|---|
| 1 | 95° C. | 5′ |
| 30 | 95° C. | 30″ |
| | 60° C. | 30″ |
| | 72° C. | 30″ |
| 1 | 72° c. | 7′ |

The qualitative amplification reaction was carried out with the following oligonucleotides that amplify the region comprised between −860 and −620 from the COX-2 gene transcription start site, comprising the −765 polymorphism:

| | |
|---|---|
| Cox-F: 5′ CCGCTTCCTTTGTCCATCAG 3′ | (SEQ. ID NO 3) |
| Cox-R: 5′ GCTATGTACACTGAAGGTAGC 3′ | (SEQ. ID NO 4) |

Numbers on the COX-2 gene sequence refer to the GenBank accession number AF276953.

The amplified segment, consisting of 231 bases, was then digested by enzymatic restriction for at least three hours with the enzyme Fau I that recognizes the consensus region CCCGCC. This enzymze cleaves only the wild type sequence carrying the G nucleotide at position −765, producing two fragments of 124 and 107 bp, respectively. It did not cut the mutated sequence carrying the C nucleotide at position −765, thus allowing the genotyping relatively to the polymorphism at this position.

The fragments generated by digestion were separated by agarose or acrylamide gel electrophoresis and recognized by RFLP.

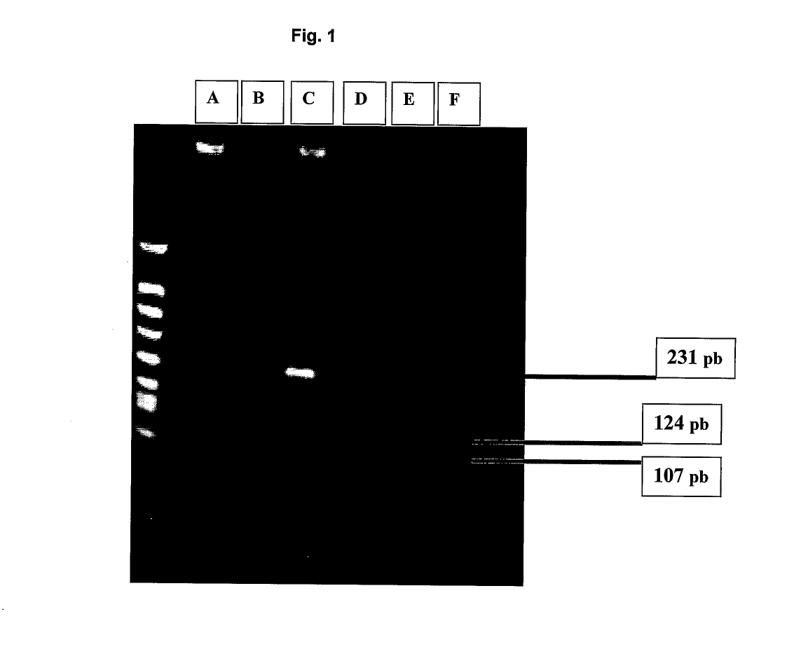

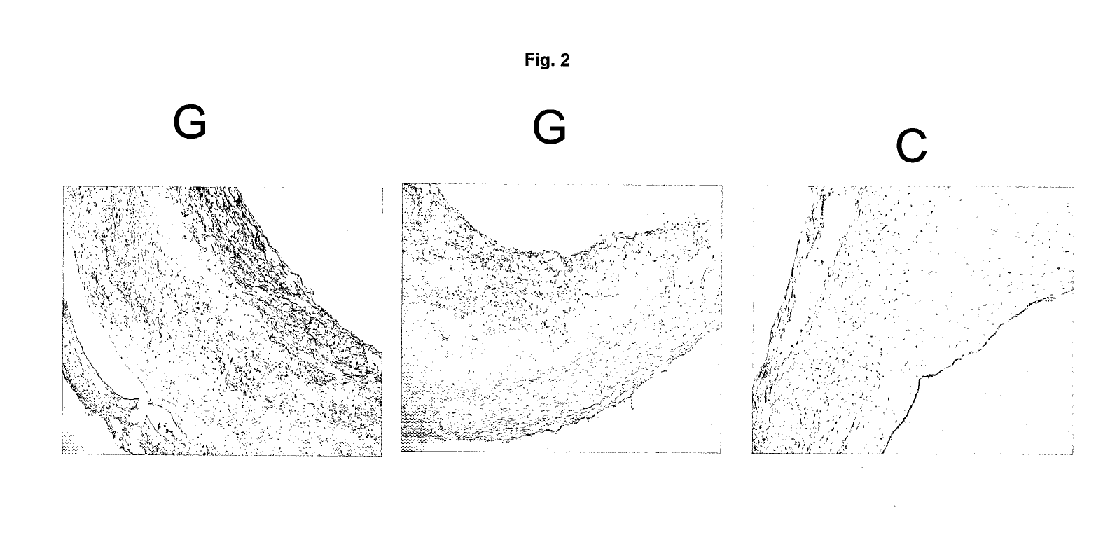

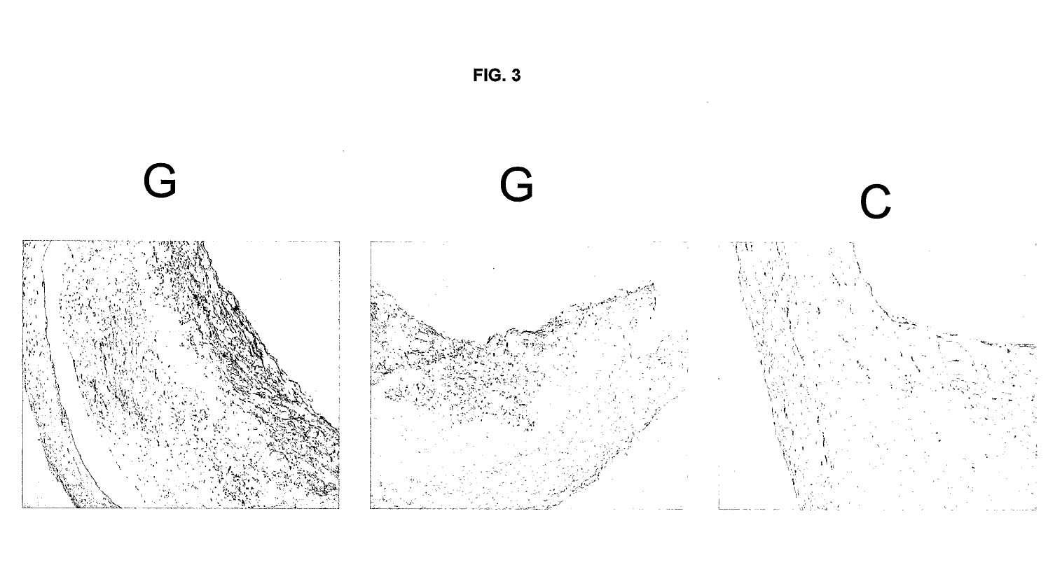

For visualization of the bands, the gel was stained with an ethidium bromide solution (0.5 μg/ml), de-stained in saline solution, and then irradiated with a UV ray source and photographed with standard detection systems. FIG. 1 shows an example of restriction pattern obtained after Fau I digestion in subjects carrying various polymorphisms.

### Example 2

### Sample Characterization

The clinical characteristics of the sample comprising 1441 subjects are summarized in Table 1.

TABLE 1
CHARACTERISTICS OF THE INDIVIDUALS SUBJECTED TO THE
STUDY.

| | PATIENTS | CONTROLS |
|---|---|---|
| Variable | (n = 864) | (n = 555) |
| Age (years) | 62.7 ± 10 | 63 ± 9 |
| Male/Female (%) | 67/33 | 65/35 |
| Subjects with: | | |
| Hypercholesterolemy, n (%) | 449 (52) | 283 (51) |
| Hypertension, n (%) | 380 (44) | 255 (46) |
| Diabetes, n (%) | 156 (18) | 105 (19) |
| Cigarette Smoking, n (%) | 311 (36) | 194 (35) |
| Body Mass Index (BMI) | 27 ± 6 | 27 ± 7 |
| NSAID or glucocorticoid treatment | 0 | 0 |
| CAD proven by angiography (n = 750)(%) | 76.2 | 76.8 |
| Single vessel pathology (%): | 30.2 | 29 |
| Double vessel Pathology (%): | 23.3 | 23.9 |
| Triple vessel Pathology (%): | 22.7 | 22.1 |
| Severity of Stenosis ICA (n = 232) | | |
| Mean ± St. Dev. (%) | 74 ± 5 | 74 ± 6 |
| Interval (%) | 70-92 | 70-91 |

NSAID = non-steroidal anti-inflammatory drugs.
CAD = coronaropathy.
ICA = internal carotid artery.

The sample did not show significantly different incidences of conventional risk factors for cardiovascular pathologies, among which: smoking habit, obesity, hypertension, diabetes and hypercholesterolemy.

The patients suffered myocardial or cerebral infarction or ischaemia only connected to the rupture of atherosclerotic plaques. Instead patients with atrial fibrillation or valvular dysfunction were excluded from the study.

The controls were recruited among subjects hospitalized for non-cardiovascular pathologies, and in particular for pathologies different from: myocardial infarction, unstable angina, TIA or stroke.

An informed consent was been obtained from all study sample subjects before each examination and the study was approved by the local ethical committee.

### Example 3

### Sample Genotyping

The presence of G→C polymorphism in the sample was assessed according to the method of the invention, as described in example 1. Data of sample genotyping are summarized in table 2.

TABLE 2
DISTRIBUTION AND MULTIVARIATE LOGISTIC REGRESSION ANALYSIS OF COX-2
VARIANTS CARRIED OUT BETWEEN CONTROLS AND PATIENTS WITH
MYOCARDIAL INFARCTION OR ICTUS..

| | DISTRIBUTION | | RATIO | | | |
|---|---|---|---|---|---|---|
| GENOTYPE | CONTROLS | CASES | CONTROLS/CASES | SIGNIFICANCE | ODDS RATIO* | 95% CI** |

Total Subjects (n = 1441)

| | | | | | | |
|---|---|---|---|---|---|---|
| GG | 50.7% | 81% | 0.62 | P < 0.0001 | | |
| GC | 43.1% | 17.9% | 2.4 | P < 0.0001 | 0.45 | 0.36-0.68 |
| CC | 6.2% | 1.1% | 5.63 | P < 0.05 | 0.34 | 0.24-0.54 |

Subjects older than 70 years (n = 257; 112 controls e 145 cases)

| | | | | | | |
|---|---|---|---|---|---|---|
| GG | 51.3% | 89.2% | 0.57 | P < 0.0001 | | |
| GC | 41.2% | 9.7% | 4.2 | P < 0.0001 | 0.41 | 0.33-0.78 |
| CC | 7.5% | 1.1% | 6.8 | P < 0.01 | 0.31 | 0.21-0.63 |

Subjects with a 1° degree relative with previous IMA*** or stroke (n = 187; 84 controls and 103 cases)

| | | | | | | |
|---|---|---|---|---|---|---|
| GG | 47.9% | 88.9% | 0.54 | P < 0.0001 | | |
| GC | 44.3% | 10.3% | 4.3 | P < 0.0001 | 0.39 | 0.29-0.64 |
| CC | 7.8% | 0.8% | 9.75 | P < 0.005 | 0.28 | 0.22-0.52 |

*The probability ratios refer to the overall risk of myocardial infarction and ictus among subjects heterozygous or homozygous for the G-C - 765 polymophism, as compared to homozygous for the G-G - 765 polymorphism.
**CI = Confidence interval.
***IMA = acute myocardial infarction.

As shown in table 2, the frequency of the G→C −765 mutation is approximately 2.4 fold higher in controls than in patients (43.1% vs 17.9%, p<0.0001) and the homozigosity for C/C is approximately 5.63 fold higher in controls than in patients (6.2% vs 1.1%, p<0.0001).

In the subgroup of 187 patients who had a first degree relative suffering myocardial infarction or ictus, the presence of C at position −765 turned out to be 10.3%, hence 4.3 fold less than in controls.

No difference in frequency of the allele carrying the C polymorphism is detected within clinical subgroups of patients who suffered myocardial infarction or ictus (17.5 vs 17.7).

### Example 4

### Characterization of Atherosclerotic Plaques

COX-2 expression was evaluated in atherosclerotic plaques of 232 patients that were subjected to endarterectomy during the study. Sections and immunohistochemical staining of atherosclerotic plaques are carried out as described in Cipollone et al., Circulation 2001. Specific staining for the COX-2 enzyme was more abundant in carotid plaques of carriers of the G −765 polymorphism (see FIG. 2).

The plaque sections were placed on glass slides pre-treated with polylysine, fixed with cold acetone for 10 minutes and washed 2 times with PBS buffer. The endogenous peroxidase activity is blocked by incubating the glass slides for 5 minutes in 3% H₂0₂ solution. After two PBS washes of 15 minutes each, the slides were saturated for 30 minutes with “blocking solution” (PBS containing 1% BSA) and subsequently incubated for 60 minutes with primary antibodies diluted in blocking solution. The antibodies are COX-2 specific monoclonals, MMP-2 specific monoclonal antibodies and monoclonal antibodies specific for MMP-9 (Cayman Chemical, Ann Arbor, Mich.), respectively for the enzyme COX-2 and for metalloprotease. Subsequently, the glass slides were washed twice with PBS and incubated for 30 minutes with secondary antibody conjugated to biotin (DAKO LSAB2 System, Peroxidase, Dako Corporation, Carpinteria, Calif.). After 2 additional PBS washes, the tissue samples were treated for 30 minutes with 100μ of a solution containing Streptavidin conjugated to the enzyme peroxidase. After 2 PBS washes, glass slides were incubated with 0.05% chromogenic substrate diaminobenzidine (DAB) for 10 minutes and washed again with PBS.

The COX-2 level was particularly high in macrophages, and this result was confirmed also by western-blot.

The specific staining for metalloproteinases 2 and 9 (MMP2 and MMP9) was evaluated by both immunohistochemistry (see FIG. 3) and western-blot. As it can be inferred from the figures showing the results of immunohistochemistry, the expression of these two enzymes was also higher in the plaques of carriers of G polymorphism. Moreover the zymography on extracts prepared from these plaques demonstrates that the two enzymes were in the active form.

### Example 5

### Assessment of the Activity of Monocytes/Macrophages In Vitro

Monocytes isolated from patients were treated in vitro with LPS, oxLDL, angiotensin II and AGE (advanced glycosylation endproducts). As it is evidentiated in FIG. 3, all the stimuli determined an increase of the synthesis of COX-2, MMP-2 and MMP-9 in monocytes of patients carrying the allele G/G −765 compared to those carrying the C polymorphism both in homozygous and heterozygous configurations. Macrophages infiltrating the plaques were isolated as described in de Vries et al. Arterioscier. Thromb. Vasc. Biol. 1999, 19:638-645. Similar results were observed in macrophages.

1. A method for assessing in vitro the predisposition of a subject to develop cardiovascular pathologies, comprising identifying the nucleotide corresponding to position 436 of seq IDN1 (COX-2 gene PROMOTER) on a sample of genomic DNA of said subject.

2. The method according to claim 1, where the genomic DNA is extracted from cells of such subject, derived from blood samples, saliva, biopsies, urine, human tissue.

3. The method according to claim 2, where said cardiovascular pathologies are caused by or associated with rupture of an atherosclerotic plaque.

4. The method according to claim 1, wherein said cardiovascular pathologies are coronaropathies, pathologies of carotid arteries, myocardial infarction, angina pectoris, acute coronary syndromes, myocardial revascularization by means of coronary by-pass or angioplasty, stroke, transient ischemic attack (TIA), peripheral arteriopathy, trombophylic syndromes.

5. The method according to claim 4, wherein said identification is carried out by one of the following techniques: sequencing, endonuclease digestion with restriction enzymes, selective hybridization with oligonucleotides specific for polymorphism at position −765 of the human COX-2 gene promoter, single strand conformational polymorphism (SSCP), DGGE, Fluorescence assisted mismatch analysis (FAMA), heteroduplex analysis, Real Time PCR.

6. The method according to claim 5, wherein said identification is carried out by endonuclease digestion with restriction enzymes.

7. The method according to claim 6, comprising the following steps:
extracting genomic DNA from a biological sample of the subject,
amplifying by means of Polymerase Chain Reaction with oligonucleotides or primers suitable for amplification of a DNA fragment comprising position −765,
enzymatically digesting such amplified fragment with a restriction enzyme selected from: Fau I and Aci I
electrophoretically separating the restriction mixture comprising the restriction fragments or of the undigested amplified fragment, or of both,
analyzing the restriction profile generated after visualization of DNA.

8. The method according to claim 7, wherein said amplifying is carried out with oligonucleotides having sequences at least partially identical to sequences ID NO 3 and ID NO 4 and the amplified fragment is digested with the restriction enzyme Fau I.

9. The method according to claim 8, wherein said amplifying is carried out with oligonucleotides having sequence SEQ. ID NO 3 and 4.

10. The method according to claim 1, wherein the presence of a cytosine (C) at position 436 of SEQ ID NO: 1, in at least one DNA allele of such subject, indicates a lower risk to predisposition to cardiovascular diseases than the risk associated to the presence of a guanosine (G) in position 436 on both alleles.

11. A kit for carrying out the method according to claim 1.

12. The kit according to claim 11, comprising at least one of the following oligonucleotides: an oligonucleotide comprising at least 10 consecutive nucleotides of seq ID NO 3, an oligonucleotide comprising at least consecutive nucleotides of seq ID NO 4 and optionally one restriction enzyme selected from: Fau I and Aci I.

13. The kit according to claim 12, comprising the oligonucleotide with sequence ID NO 3 and the oligonucleotide with sequence ID NO 4, the Fau I restriction enzyme and optionally one molecular weight DNA standard.

14. A prognostic method for a cardiovascular pathology selected from the group consisting of: coronaropathies, pathologies of carotid arteries, myocardial infarction, angina pectoris, acute coronary syndromes, myocardial revascularization by means of coronary by-pass or angioplasty, stroke, transient ischemic attack (TIA), peripheral arteriopathy, and trombophilic syndromes, comprising genotyping of nucleotide at position 436 of SEQ ID NO: 1 (COX-2 gene promotor).

15. A method of assessing the sensitivity to therapy with non steroidal anti-inflammatory drugs (NSAIDs) comprising genotyping of nucleotide at position 436 of SEQ ID NO: 1 (COX-2 gene promotor.

16. The kit for carrying out the method according to claim 10.

17. The method according to claim 16 wherein the presence of a cytosine (C) at position 436 of SEQ ID NO: 1, in at least one DNA allele of such subject, indicates a lower sensitivity to therapy with non steroidal anti-inflammatory drugs (NSAIDs) than the presence of a guanosine (G) in position 436 on both alleles.

18. A kit for assessing the sensitivity to therapy with non steroidal anti-inflammatory drugs (NSAIDs) comprising genotyping a nucleotide at position 436 of SEQ ID NO: 1 (COX-2 gene promotor) with suitable oligonucleotides.

19. A kit according to claim 18 comprising the oligonucleotides having SEQ ID NO: 3 and SEQ ID NO: 4.


SEQUENCE LISTING (4 sequences)

SEQ ID NO: 1 - DNA, 1400 bp, Homo sapiens
  CDS (1335)..(1400)
ttccagctgtcaaaatctcccttccatctaattaattcctcatccaactatgttccaaaacgagaatagaaaattagccccaataagcccaggcaactgaaaagtaaatgctatgttgtactttgatccatggtcacaactcataatcttggaaaagtggacagaaaagacaaaagagtgaactttaaaactcgaatttattttaccagtatctcctatgaagggctagtaaccaaaataatccacgcatcagggagagaaatgccttaaggcatacgttttggacatttagcgtccctgcaaattctggccatcgccgcttcctttgtccatcagaaggcaggaaactttatattggtgacccgtggagctcacattaactatttacagggtaactgcttaggaccagtattatgaggagaatttacctttcccgcctctctttccaagaaacaaggagggggtgaaggtacggagaacagtatttcttctgttgaaagcaacttagctacaaagataaattacagctatgtacactgaaggtagctatttcattccacaaaataagagttttttaaaaagctatgtatgtatgtgctgcatatagagcagatatacagcctattaagcgtcgtcactaaaacataaaacatgtcagcctttcttaaccttactcgccccagtctgtcccgacgtgacttcctcgaccctctaaagacgtacagaccagacacggcggcggcggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaatttgtggggggtacgaaaaggcggaaagaaacagtcatttcgtcacatgggcttggttttcagtcttataaaaaggaaggttctctcggttagcgaccaattgtcatacgacttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctgacgctcagccatacaggtgagtacctggcg

SEQ ID NO: 2 - protein, 22 aa, Homo sapiens
MLARALLLCAVLTLSHTGEYLA

SEQ ID NO: 3 - DNA, 20 bp, Artificial sequence
  primer_bind (1)..(20): synthetic primer
ccgcttcctttgtccatcag

SEQ ID NO: 4 - DNA, 21 bp, Artificial sequence
  primer_bind (1)..(21): synthetic primer
gctatgtacactgaaggtagc